SEQUENCE LISTING (3 sequences)

SEQ ID NO: 1 - DNA, 34 bp, Primer-rat pancreas cell line BSp73-AS
ctcggaagcttagccaccaaccagccaagatgcc

SEQ ID NO: 2 - DNA, 68 bp, Primer-rat pancreas cell line BSp73-AS
gccatgaattcctagcccgcatagtcaggaacatcgtatgggtacatggcctcaaactcgtcgatgcg

SEQ ID NO: 3 - DNA, 68 bp, Primer-rat pancreas cell line BSp73-AS
gccatgaattcctagcccgcatagtcaggaatatcgtatgggtactgggccttcatctgctgcacctc

(19) United States
(12) Patent Application Publication
(10) Pub. No.: US 2004/0072171 A1
(43) Pub. Date: Apr. 15, 2004

(54) Method for identifying compounds for modulating the activity of a tumor suppressor protein

(76) Inventors: Helen Morrison, Karlsruhe (DE); Helmut Ponta, Weingarten (DE); Peter Herrlich, Karlsruhe (DE)

Correspondence Address: Friedrich Kueffner, Suite 910, 317 Madison Avenue, New York, NY, 10017, US

(21) Appl. No.: 10/296,424

(22) PCT Filed: May 22, 2001
(86) PCT No.: PCT/EP01/05842

(30) Foreign Application Priority Data
May 30, 2000 (DE) 100 26 833.1

(51) Int. Cl. C12Q 1/68; G01N 33/574

(52) U.S. Cl. 435/6; 435/7.23

(57) ABSTRACT

The present invention relates to a method for identifying compounds which modify the activity of the intracellular tumor suppressor gene nf2, where the activity of the protein NF2, which is encoded by the gene nf2, is modified by an extracellular interaction of the compounds with the cell surface protein CD44.

The present invention relates to a method for identifying compounds in the field of tumor suppressors.

The ability of cells to stop proliferating when the space assigned to them is full is an important property of cells of a multi-celled organism. This process, which is termed contact inhibition, has already been known for a long time. The signal to which the cells respond during the process of contact inhibition might be the contact of the cells with one another or the contact between the cells and the extracellular matrix. The process of contact inhibition requires the presence of one or more surface sensors which communicate with the nucleus and halt the cell cycle in the G1 phase, thus bringing about inhibition of growth. Normal cells multiply when cultured in vitro until they out the surface of the culture vessel and then stop in the G1/G0 phase when a thick cell monolayer has formed (contact inhibition). A further phenomenon of their halted growth is their inability of forming colonies when embedded in a sufficiently dense matrix (soft agar).

The entirety of mechanisms owing to which cell growth stops is as yet largely unknown. The loss of contact inhibition and colony formation in soft agar are properties which are characteristic of cancer cells.

Type II neurofibromatosis (NF2) is a hereditary multi-tumor disease. Worldwide, one in 40 000 people suffers from this disease. The gene in question was identified as nf2, which encodes the NF2 gene product otherwise known as merlin (“merlin” means moesin-ezrin-radixin-like protein, also termed schwannomin). A typical feature of NF2 is the occurrence of bilateral tumors of the eighth cranial nerve, which are termed schwannomas. Moreover, NF2 patients suffer from ependymomas, meningiomas, spinal schwannomas and opacification of the phacocyst. Mice in which both merlin alleles are impaired are not viable. Heterozygous mice which only lack one allele develop aggressive tumors whose cells have lost the healthy allele. The loss of heterozygosity at the nf2 locus is also found in tumors of the spontaneously occurring schwannomas, meningiomas and ependymomas (Evans D. G. et al., 1992; J.Med. Genet. 29, 841-846; Jacoby L. B. et al., 1996; Genes Chromosomes Cancer 17, 45-55; Twist E. C. et al., 1994, Hum. Mol. Genet. 3, 147-151). Biachi and Cheng report that nf2 mutations are observed in tumors which are not associated with NF2 disease, including mesotheliomas and mammary carcinomas (Bianchi A. B. et al., 1994; Nat. Genet. 6, 185-192; Cheng et al., 1999, Genes Chromosomes Cancer 24, 238-242).

McClatchey et al. have demonstrated on mice with specific nf2 mutations that merlin acts as a tumor suppressor on a variety of cell types, also outside the nervous system (McClatchey et al., 1998, Genes Dev. 12, 1121-1133). A lack of the NF2 protein merlin results in the metastatic spread of tumors. These and other data show that merlin has a general function as a tumor suppressor (Rouleau et al., 1987, Nature 329, 246-248; review by Gusella et al., 1999, Biochim. Biophys. Acta 1423-M29-36).

The N-terminal half of merlin shows a high degree of homology with the ezrin-radixin-moesin (ERM) family of the band-4.1-related proteins, which are not tumor suppressors (review by Gusella et al., 1999). According to Tsukita and Yonemura (1997, Trends Biochem. Sci. 22, 53-58), the ERM proteins connect the actin cytoskeleton and transmembrane proteins. The N-terminal domains of the ERM proteins bind in vitro to a sequence motif of charged amino acids of the cytoplasmic moiety of the transmembrane proteins CD44, CD43 and ICAM-2, while the C termini of the ERM proteins are associated with actin (Yonemura et al., 1998, J. Cell. Biol. 140, 885-895).

Owing to the similarity between the ERM proteins and merlin with regard to the N-terminal sequence, merlin is capable either of sharing the functional properties of the ERM proteins and/or of competing with the ERM proteins for a shared interaction site. Using microscopic methods, Sainio et al. (1997, J. Cell. Sci. 110, 2249-2260) found colocalization of one of the transmembrane proteins, viz. CD44, with ezrin and merlin. Moreover, it has been found that, depending on hitherto unknown conditions, merlin is also located in the cytoplasm (LaJeunesse et al., 1998, J. Cell. Biol. 141, 1589-1599).

The transmembrane proteins CD44, CD43 and ICAM-2, to which proteins of the ERM family bind, can exhibit different ligands on the other side of the plasma membrane, i.e. the extracellular region. DE 40 14 510 describes antibodies which bind to variant CD44 surface proteins. Sleeman et al. (1997, J. Biol., Chem. 272, 31837-31844) describes CD44 as a receptor for glycosaminoglycans such as, for example, hyaluronate. According to Sherman et al. (1995, J. Neuro-Oncology 26, 171-184) CD44 is the most important hyaluronate receptor in some cell lines.

Not only the ERM proteins, but also merlin, are modified by phosphorylation and therefore probably exist in two functionally different states (Matsui et al., 1998; Shaw et al., 1998b; see also review by Hall, 1998). The N- and C-terminal domains of the ERM proteins are capable of intramolecular association. It is assumed that phosphorylation of these proteins has an effect on the association of the N and C termini and influences the interaction of the ERM proteins with certain binding partners in the cell. Neither the factors which regulate phosphorylation nor the role which merlin modifications play during growth regulation are known as yet.

However, it appears that merlin acts as an antiproliferative protein in a plurality of cell types. Experiments in which merlin was transfected into rat schwannomas (Sherman et al., 1997, Oncogene 15, 2505-2509) or NIH3T3 cells (Lutchman et al., 1995, Cancer Res. 55, 2270-2274; Tikoo et al., 1994, J. Biol. Che. 269, 23387-23390) have demonstrated that merlin inhibits cell proliferation and counteracts Ras transformation. Since most of the NF2 patients carry merlin mutations in the N-terminal half (Koga et al., 1998, Oncogene 17, 801-810), the N terminus appears to be of particular importance for the control of proliferation.

Sherman et al. demonstrated that overexpression of merlin in the experimental rat schwannoma cell line RT4-D6P2T, both in vitro and in vivo, inhibits cell growth. However, after several cell divisions, this potent growth-inhibitory effect results in the loss of merlin expression. This is why the mechanistic biochemical analysis of the function of merlin has not been possible as yet.

It is therefore obvious that merlin (NF2) plays a decisive role as tumor suppressor in cell proliferation and the control of growth. Since merlin inhibits the proliferation of a variety of cell types, it can be employed as a broad-range tumor suppressant. It is therefore highly important from the medical point of view, in particular for cancer research, to identify compounds which have a positive effect on the activity of merlin as tumor suppressant. Owing to the association of the tumor suppressant function with the transmembrane protein CD44, which association is described in the present invention, suitable candidates are not only drugs which act intracellularly, but also, in particular, molecules which target extracellularly. The identification of these extracellular compounds requires the provision of a system with an accurately defined signal pathway. This is another object of the present invention.

The present invention relates to a method for identifying compounds which modify the activity of the intracellular tumor suppressor gene nf2, where the activity of the protein NF2, which is encoded by the gene nf2, is modified by an extracellular interaction of the compounds with the cell surface protein CD44. The method is furthermore characterized in that the expression of the gene nf2 or the activity of the protein NF2 is increased.

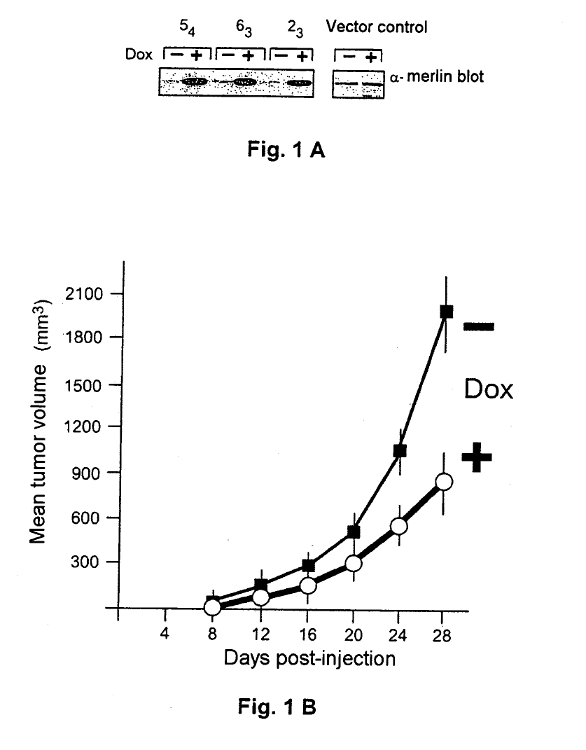

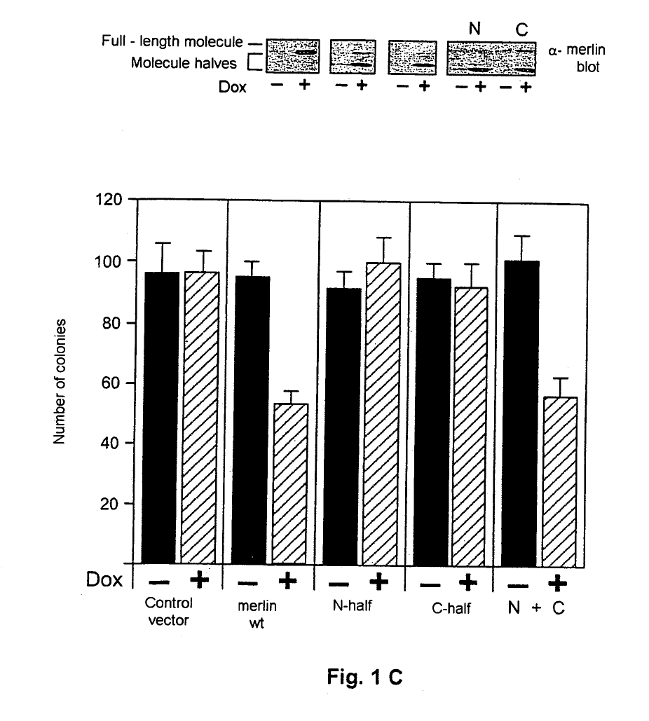

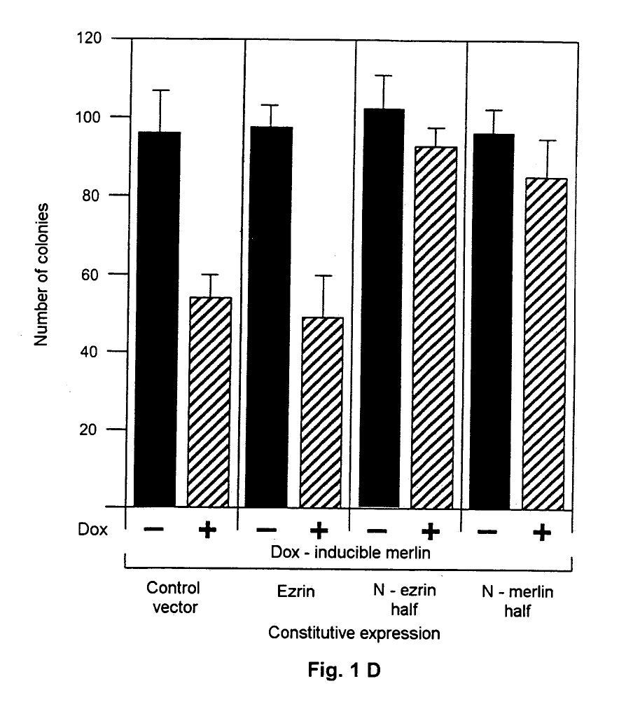

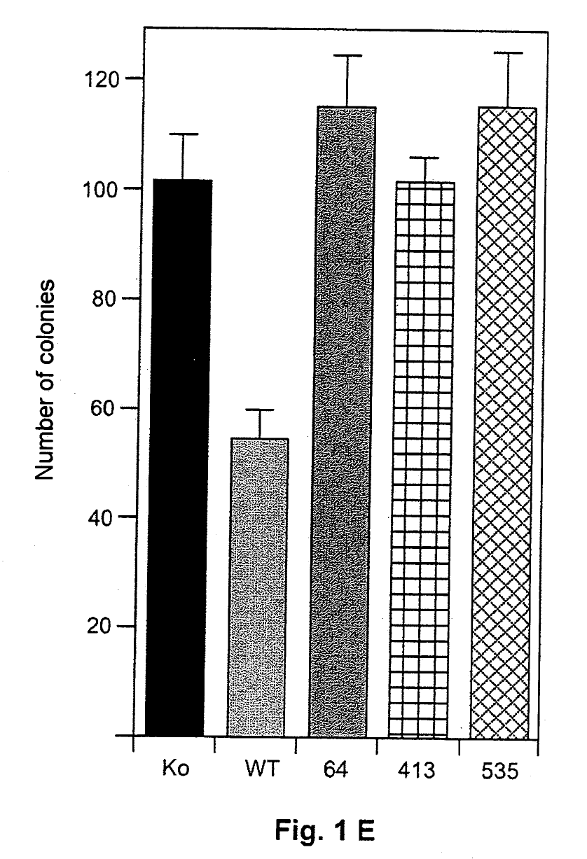

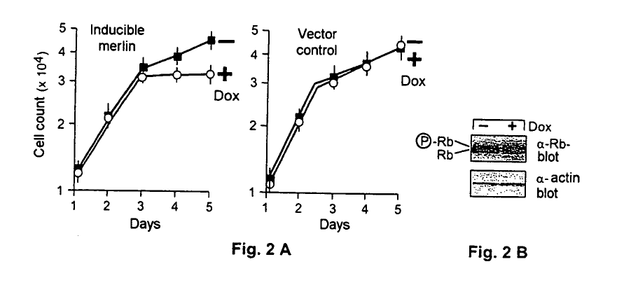

A system according to the invention with an accurately defined signal pathway was developed in order to make possible an induced continuous expression of merlin. This system is characterized in that clones of the rat schwannoma cell line RT4-D6P2T which comprise not only an expression vector encoding a reverse-tet repressor, but also a merlin cDNA plasmid under the control of a tet repressor recognition sequence, are used. The reverse-tet repressor acts as transcription activator (Gossen et al. 1995, Proc. Natl. Acad. Sci. USA, 89, 5547-51). The low basal expression of merlin, of the clones, is induced greatly by the addition of doxycyclin. It has been demonstrated that the induction of merlin inhibits tumor growth in vivo and in vitro. The results are compiled in FIGS. 1A, B, C and E and in FIG. 2A.

These results show that the induction of merlin expression in the rat schwannoma system suppresses tumor growth in vivo and in vitro and that this system is therefore suitable for studying the function of merlin and for identifying compounds with a regulatory effect.

Furthermore, the biochemical function of merlin can be subjected to an accurate mechanistic analysis using the rat schwannoma system according to the invention.

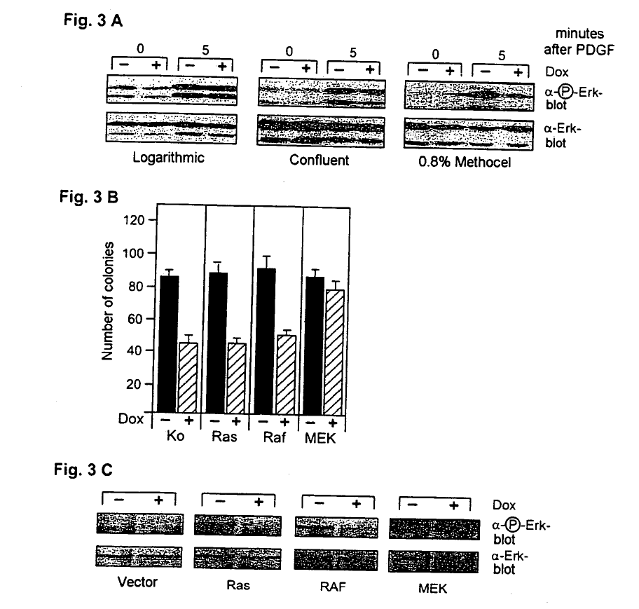

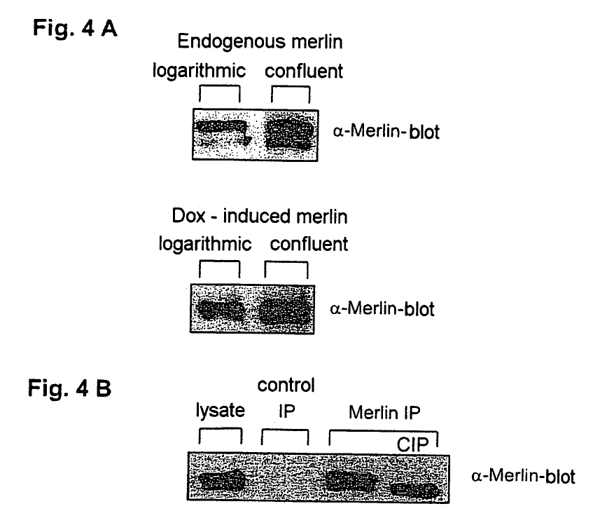

The invention also relates to the steps in the mechanism of action of merlin as tumor suppressor. The loss of contact inhibition in tumor cells is observed frequently and is connected with the activation of certain oncogene-driven signal transmission cascades. The rat schwannoma system can be used to demonstrate that merlin influences the Ras MAP kinase signal tranduction cascade in order to reinstitute contact inhibition. To this end, the effect of merlin on the direct activation of the components of this signal transduction pathway is studied as a reaction to stimulation by a growth factor, preferably the PDGF-dependent activation of Erk (serin-threonin kinase) (FIG. 3A). In the case of doxycyclin-induced expression of merlin, reduced activation of Erk following PDGF treatment is observed, but only in confluent cell cultures. In cell clones with inducible merlin expression, constitutively active Ras, Raf or MEK can be assayed for colony formation in soft agar and for Erk activation in the presence or absence of doxycyclin. This method allows the determination of the exact site where merlin engages in the signal transduction cascade. The results of FIG. 3 show that merlin interferes with signal transduction the level of, or downstream, of Raf, but before MEK (tyrosine-threonine-kinase) activation. This is confirmed by the results in FIG. 4. An MEK activation inhibitor has the same effect as merlin, i.e. the inhibition of the Ras-MEK pathway, the induction of p27 and halting of the cell division cycle.

The invention also encompasses the use of the rat schwannoma system according to the invention in elucidating the role of the N- and C-terminal domains of merlin, since mutations in the N terminus are the most frequent ones in NF2 patents. When the molecule halves are mixed in vitro, they are capable of reassociation. Studies with tet-inducible constructs which encode either the N-terminal half or the C-terminal half of merlin and which are stably introduced into the schwannoma cell system have demonstrated that reassociation also takes place in vivo. Despite the two constructs being expressed as two separate peptides, their coexpression in the same cell restitutes the full merlin activity, that is to say the two domains, in this case molecule halves, can be associated within the cells (FIGS. 1C, D and FIG. 2B).

The rat schwannoma system according to the invention, in which the expression of merlin can be induced in a controlled fashion, makes it possible to study a further step in the mechanism of the effect of merlin on the proliferation of cells. In cultured schwannoma cells, uninduced cells continue to proliferate, upon reaching confluence, in the manner which is typical of tumor cells. They obey contact inhibition signals insufficiently or not at all. In the case of induced cells, i.e. cells in which merlin is expressed at a high level, induced by doxycyclin, confluence brings about a halt in growth and an increased number of cells in the G1 phase. Accordingly, these cells resemble transformed cells which obey contact inhibition signals (FIG. 2B).

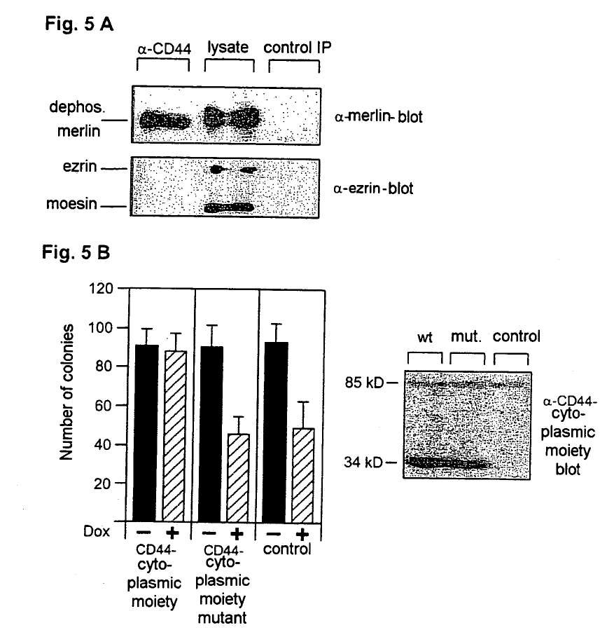

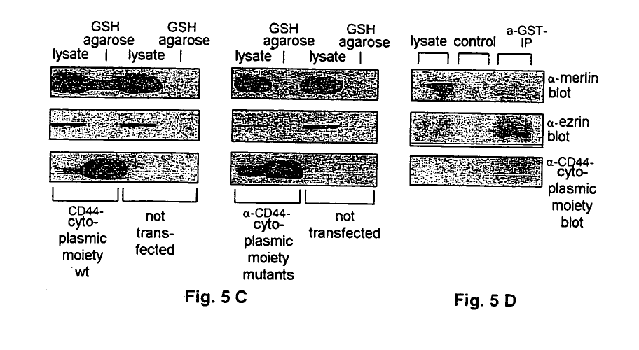

Thus, merlin exists in two different forms, viz. in growth-suppressing form at high cell density and in inactive form at low cell density. Induction of the active form therefore leads to the consequences which can normally be observed in untransformed cells. These consequences are the accumulation of hypophosphorylated retinoblastoma protein Rb and an increase in the level of cell cycle inhibitors p21 and p27 in the nucleus, which reduces the incorporation of thymidin (FIG. 5A) (St. Croix et al., J. Cell Biol. 142, 557-71, 1998). This is to say that merlin “interferes” with the step in the signal cascade which influences the progression of the cell division cycle.

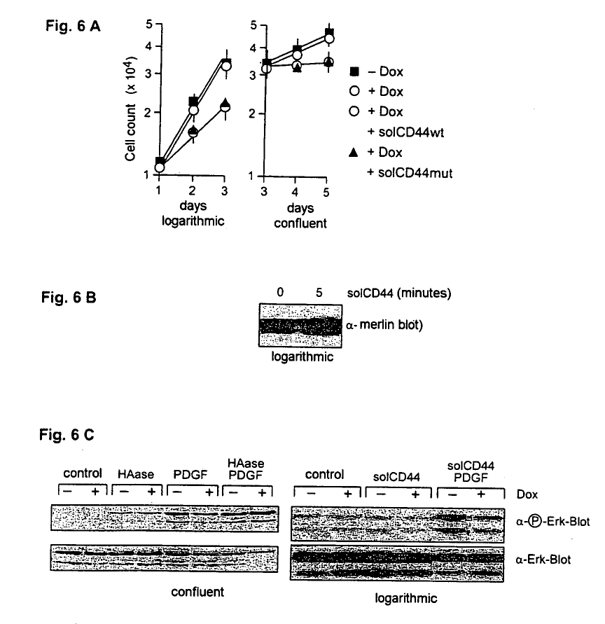

Moreover, merlin shows structural differences which correlate with the function and which depend on the cell density, i.e. merlin is activated by post-translational modifications. Electrophoretic separation and Western blots of RT4-D6P2T cell lysates separate merlin into two bands (FIG. 6A). In the case of nonconfluent cultures, a band which migrates more slowly predominates, while a second band which migrates more rapidly is only observed in confluent cultures. Since the band which migrates more slowly disappears when the lysates are digested with calf intestine phosphatase, the band which migrates more rapidly is a hypophosphorylated form of merlin (FIG. 6B). However, the hypophosphorylated molecule can only be observed in lysates of confluent cultures or of cells grown in soft agar, even when the expression of merlin is induced at a high level. These results mean that the hypophosphorylated form of merlin acts as growth suppressor and that conditions which promote contact inhibition inhibit the phosphorylation and function of merlin.

The invention also relates to a method for identifying compounds which modify the activity of the intracellular NF2 protein wherein NF2 is activated according to the invention by dephosphorylation of the protein.

Further studies within the scope of the present invention have demonstrated that merlin is synthesized as an inactive molecule. Activation requires the conditions of a confluent cell culture. Since the effect of merlin as growth suppressor and its phosphorylation status depend on cell density, the function of merlin can be modified as a response to interactions between the cells and/or between the cell and the matrix. Activation by an extracellular stimulus requires signal transduction across the plasma membrane, for example via a transmembrane protein.

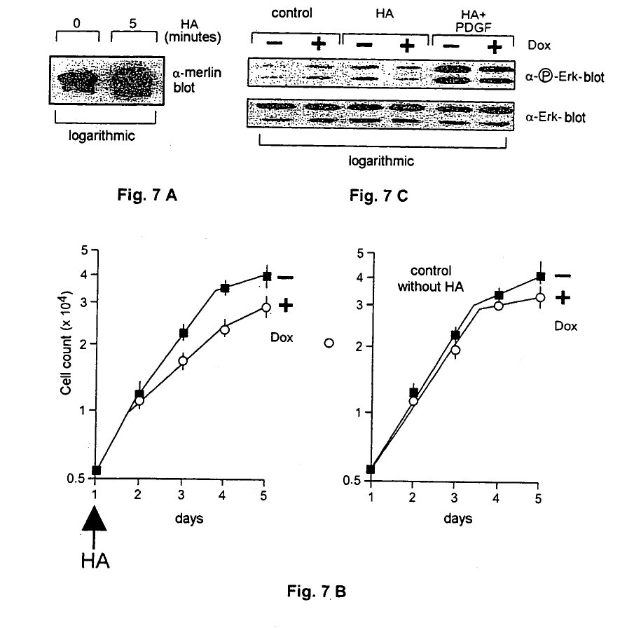

As demonstrated by Sainio et al., merlin interacts in vitro with the cytoplasmic moiety of CD44. Coimmuno-precipitation experiments within the scope of the present invention have demonstrated that, using the CD44-specific antibody 5G8, merlin is only precipitated with CD44 (Sleeman et al., 1996, Cancer Res. 56, 3134-3141) in a coimmunoprecipitation when the cells reach confluence (FIG. 7A; not shown for the cells in the log phase, but see FIG. 7D). This means that only the hypophosphorylated form of merlin associates with CD44. The above interactions between CD44 and merlin at the cell membrane is required if merlin is to inhibit cell proliferation. This is demonstrated by experiments with stable clones, prepared from the schwannoma cells which can be induced according to the invention, which overexpress the cytoplasmic moiety of CD44 in fusion with GST (Smith and Johnson, 1988, Gene 67, 31) as soluble cytoplasma peptide. In these clones, expression of the cytoplasma fusion protein is 20 times more pronounced than that of the endogenous CD44 (FIG. 7B). The CD44-cytoplasmic-moiety/GST fusion protein sequesters merlin from its activation site at the transmembrane protein CD44 and thus blocks the growth inhibition in soft agar which is caused by merlin. In contrast, the expression of GST alone has no effect on the function of merlin (FIG. 7B).

In a similar clone which expresses the CD44-cyto-plasmic-moiety/GST fusion protein with a mutation in the ERM-protein-binding domain, the growth inhibition caused by merlin is not nullified (FIG. 7B). This demonstrates that, while merlin is precipitated in an immunoprecipitation by the cytoplasmic moieties of the wild-type CD44, the cytoplasmic moieties of the mutant are ineffective (FIG. 7C).

Even though the above-described result demonstrates that merlin interacts with the ERM-protein-binding domain of CD44, it does not compete directly with ezrin for this domain. Ezrin cannot be obtained from lysates of confluent cultures by coimmunoprecipitation with CD44 (FIG. 7A). Under confluent conditions, only merlin binds to the cytoplasmic moiety of CD44 (FIG. 7C). This is shown by a study of the association of ezrin and CD44, where cells are used which stably express the CD44-cytoplasmic-moiety/GST fusion protein and where this protein is isolated by glutathione agarose (FIG. 7C) or by immunoprecipitation with an anti-GST-antibody (FIG. 7D). In contrast, ezrin associates during the exponential growth phase with CD44, while merlin does not (FIG. 7D). This means that the binding of merlin to CD44 precludes the binding of ezrin and vice versa. Moreover, the function of the two proteins depends on their interaction with CD44 (FIG. 1D). However, merlin and ezrin are not simultaneously active under the same growth conditions.

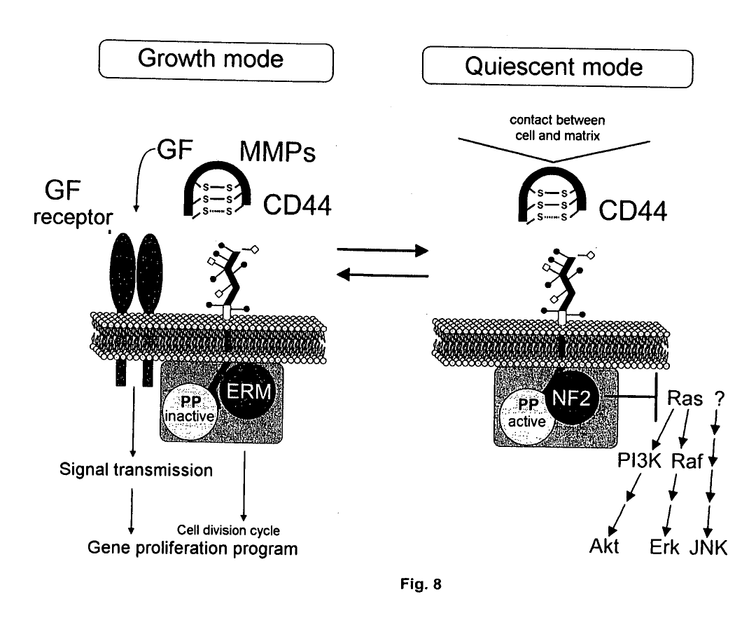

FIG. 8 is a schematic representation of the conformation of the contact inhibition complex during the logarithmic growth phase. A complex of either merlin and a phosphatase or ezrin and a protein kinase is located on the cytoplasmic side of the cells. In both cases, the two components remain close to each other. The cytoplasma complex may additionally comprise other proteins whose identity is as vet not elucidated. Various ligands may bind on the extracellular side.

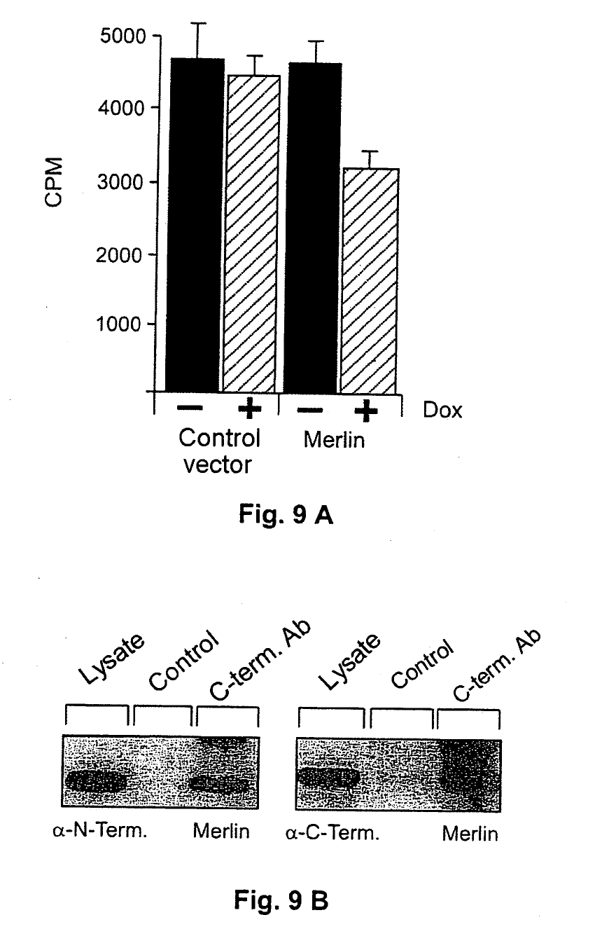

Within the cell, merlin can exist in two conformations so that it has an inhibitory effect on the cell cycle upon confluence, but allows or indeed promotes the progression of the cell division cycle during the log phase. This means that, on the one hand, the merlin complex on the interior of the cell is subject, between confluence and logarithmic growth, to a change from hypophosphorylated to hyperphosphorylated, on the other hand, CD44 ligands in the extracellular moiety determine, in accordance with the invention, the components and properties of the complex as shown in FIGS. 8 and 9.

An embodiment of the invention relates to a method for identifying compounds which modify the activity of the intracellular tumor suppressor NF2, characterized in that

a) a cell culture is equipped with a dominant oncogene,

b) this “oncogenic” cell culture is additionally equipped with a gene construct which contains a promoterless reporter gene under the control of an Ras-dependent promoter (Hofmann, 1993, Cancer Res. 53, 1516),

c) the cell culture thus obtained is provided with a compound which, owing to interaction with a cell surface protein, is potentially capable of increasing the intracellular activity of NF2,

d) a substance which can be converted by the expression product of the reporter gene is added to the cell culture thus treated,

e) the substance added in d) is, if appropriate, removed,

f) the cell culture thus treated is equipped with a suitable culture medium in order to multiply the cells, and

g) a compound of c) is indeed identified as one which increases the intracellular activity of NF2 which leads to reduced cell multiplication and/or the expression of the reporter gene.

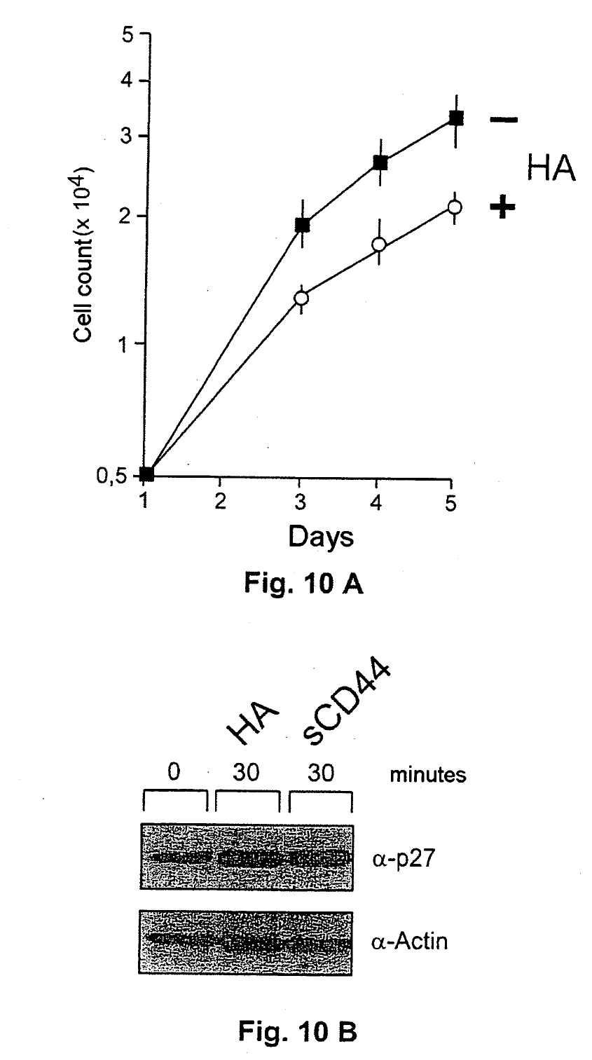

In accordance with the invention, the change in the activty of merlin is brought about by the interaction of CD44 with merlin on the intracellular side of the plasma membrane and by interaction of CD44 with ligands on the extracellular side of the plasma membrane. Prior to binding to the cell membrane, possible ligands can, by addition proteins which only scavenge the extracellular part of CD44 to the cell culture and prevent it from binding to the transmembrane protein. They can also be isolated and identified thereby. Examples of such proteins are splice variants and mutants of CD44 (CD44s). The results of the experiments with the smallest splice variant CD44s show that growth inhibition is nullified in confluent cell cultures in which the expression of merlin is induced by doxycyclin (FIG. 10). When using a CD44s mutant which lacks the glycosaminoglycan bond, in contrast, growth inhibition is retained. This means that merlin is activated by a glycosaminoglycan which binds to CD44 on the extracellular side of the membrane.

Accordingly, a variant of the method according to the invention is characterized in that a rat schwannoma system comprising clones of RT4-D6P2T cells is used, these clones comprising not only an expression vector encoding a reverse-tet repressor, but also a merlin cDNA plasmid under the control of a tet repressor recognition sequence. The basal expression of merlin in the clones is low and is greatly induced by the addition of doxycyclin.

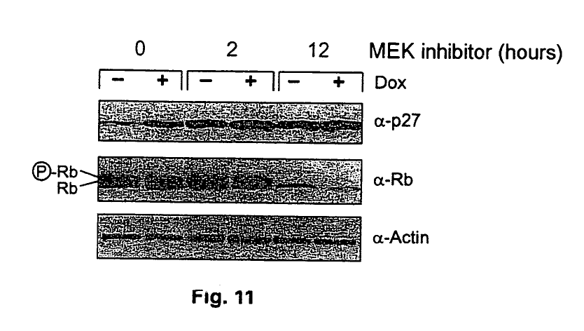

The experiments which have been described so far have demonstrated that merlin is activated by extracellular substances via interaction with CD44. Hyaluronate is a glycosaminoglycan synthesized by schwannoma cells. The cleavage of hyaluronate by hyaluronidase in a confluent culture prevents the inhibition of growth (FIG. 10). On the other hand, the results of FIG. 11 show how hyalonurate can imitate the effects of contact inhibition in cultures of logarithmically growing cells.

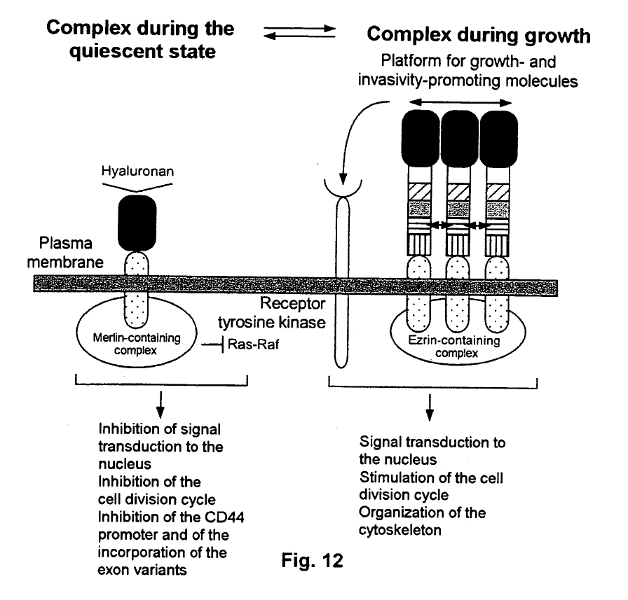

FIG. 12 shows that hyaluronate-induced activation of merlin is also observed in NIH3T3 cells, which, accordingly, leads to the induction of p21 and p27 and to delayed growth.

The present invention also relates to a method which is characterized in that, prior to the identification of compounds which modify the activity of the intracellular tumor suppressor gene nf2, a desired cell culture is tested for its suitability by the inhibition of cell growth owing to the addition of hyaluronate and/or other specific antibodies against the hyaluronate binding site on CD44.

The hyaluronate-induced activation of merlin leads to halted growth when the cultures reach a high cell density. In exponentially growing cultures, however, other ligands may exist in CD44-bound form. The results of FIG. 10 show that in cultures in the logarithmic growth phase which are treated with soluble wild-type and mutant CD44s, growth inhibition is observed. This means that cells in the log phase have attached to them a CD44 ligand which has an inhibitory effect on merlin, but which is other than hyaluronate. Removal of this inhibitory ligand by the soluble extracellular CD44 domain instantly results in dephosphorylation, i.e. the activation of merlin (FIG. 10B).

A further variant of the invention relates to a method which is characterized in that, prior to the identification of compounds which modify the activity of the intracellular tumor suppressor gene nf2, a desired cell culture was tested for its suitability by nullifying the growth inhibition in confluent cultures and/or inducing growth inhibition in cultures in the log phase by the addition of soluble CD44 proteins, comprising extracellular domains, and/or mutants thereof.

At least two different specific ligands are capable of binding to the extracellular side of CD44, viz. a ligand which is characteristic of logarithmic growth and a second ligand which is found in confluent cells and which mediates contact inhibition.

However, only very few physiologically active compounds are known as yet which regulate the activation of merlin as extracellular ligands. However, the knowledge of such ligands might be useful in tumor therapy. The compounds which activate merlin should reduce tumor growth. A screening for such CD44 ligands for identifying tumor-suppressing compounds is therefore of great medical interest.

This can be carried out with the aid of the above-described methods and systems or with other variants of the method according to the invention.

Another variant of the present invention relates to a negative assay which is characterized in that the gene RasV12 is employed as the dominant oncogene (Lowy 1993, Ann. Rev. Biochem., 62, 851). The Ras-dependent promoter employed is the promoter of the c-fos gene (Verma, 1987, Adv. Cancer Res. 49, 29) or of the human collagenase gene (Angel et al., Cell, 1987). The dominant oncogene drives the expression of the reporter gene. Under these conditions, the cells express the gene product of the reporter gene until the activation of merlin leads to the expression being switched off.

Another variant describes a method which is characterized in that a gene construct comprising a promoterless thymine kinase gene (Hilhie, 1979, Nucl. Acid Res. 7, 859) under the control of an Ras-dependent promoter is employed in step b). Moreover, this variant is characterized in that gancyclovir is employed in step d).

The present invention also relates to a variant for identifying compounds with modified activity of the intracellular tumor suppressor gene nf2, which variant is based on a positive assay. This method is characterized in that a gene construct comprising a promoterless reporter gene is employed in step b) of the method, a CD44 exon (König, 1998, EMBO 17, 2904), preferably the CD44 exon v5, being integrated into the coding region of the reporter gene. Furthermore, the method is characterized in that, owing to an increased NF2 activity caused by the compound added in step c), the incorporation of the CD44 exon into the mature mRNA of the reporter gene, which is caused by Ras, is prevented and the expression product of the reporter gene is detected specifically. The reporter gene is inserted in such a way behind the exon v5 that it can either only be read after loss of the sequence of exon v5 or, as an alternative, only in the presence of v5 in the mature RNA. This method is characterized in that a promoterless luciferase gene (De Wet, 1987, Cell Biol. 7, 725) or a promoterless green fluorescent protein is employed as reporter gene. Furthermore suitable as reporter genes are the lacZ gene, which encodes a β-galactosidase, genes encoding fluorescent proteins, for example GFP (“green fluorescence protein”) (Genbank

Accession No. GFP U 47997) or the corresponding red or blue fluorescent proteins, genes encoding specific surface antigens which are identified by antibodies or genes which encode a resistance to active substances, such as, for example, neomycin, gentamycin or hygromycin. Preferred in accordance with the invention are genes encoding fluorescent proteins. The present invention also encompasses all those selection markers which are known from the literature, have not been mentioned specifically and are suitable for the purposes of the invention.

The present invention furthermore relates to a vector for use in a method as described above which is characterized in that it comprises a promoterless reporter gene under the control of an Ras-dependent promoter and additional structures responsible for NF2-dependent, targeted splicing of exons from the derived mature mRNA of the reporter gene.

Moreover, the present invention relates to compounds which have been identified by the method according to the invention. In a variant of the present invention, these take the form of compounds which are characterized in that they are the CD44-specific antibodies IM7 or KM81. The present invention furthermore relates to these compounds which are low-molecular-weight chemical compounds, preferably other than hyaluronic acid. Moreover, the present invention relates to compounds which bind specifically to a sequence of a cell surface protein (in the present case CD44).

The present invention furthermore relates to the use of the compounds identified by the method according to the invention for the preparation of compositions for treating carcinomas.

### EXAMPLES

### 1. Growth Factors and Reagents

The growth factors and reagents were obtained from the following companies: recombinant human platelet-derived growth factor BB (Biomol, Hamburg); doxycyclin (Sigma, Diesenhofen); hyaluronate (Healon; high-molecular-weight; Pharmacia & Upjohn, Erlangen); type VI-S hyaluronidase from bovine testes (Sigma); glutathione agarose (Santa Cruz, Calif.); nonidet P-40 (NP40; Boehringer Mannheim).

### 2. Antibodies

The following polyclonal rabbit antibodies were used for detecting merlin: A-19, N-terminal epitope; C-18, C-terminal epitope (Santa Cruz). The antibodies which recognized the retinoblastoma protein (Rb) were obtained from Santa Cruz (C-15). The antibodies against the CD44-cytoplasmic moiety were prepared in accordance with standard methods and are described elsewhere. Other antibodies were: the antibody specific for phosphorylated Erk was obtained from New England Biolabs (Schwalbach), those against Erk (K23), p27 (C-19), GST (Z-5) and actin (I-19) were obtained from Santa Cruz. The ezrin-specific antibodies (E13420) were obtained from Transduction Labs (Dianova, Hamburg) and those for the hemagglutinin marker (12CA5) from Boehringer Mannheim. The CD44-specific antibody 5G8 has already been described (Sleeman et al., 1996).

### 3. Plasmid Constructs

pUHD17-1, which encodes the reverse tetracycline-dependent transactivator (rtTA), pUHC13-3, the rtTA-responsive luciferase reporter, and pUHD10-3, the rtTA-responsive cloning vector (Gossen and Bujard, 1992; Gossen et al., 1995) were kindly provided by Hermann Bujard (Heidelberg). The EcoR1 fragments which encode either the complete merlin cDNA (NF2.17) or the N-terminal half (NF2.17-N-term) or the C-terminal half (NF2.17-C-term) (Sherman et al., 1997) were subcloned into pUHD10-3. The pcDNA3-NF2 mutants 64, 413 and 535 are as described by Gutmann et al. (1999, Hum. Mol. Gen. 8, 267-285). Rat ezrin cDNA was isolated from a cDNA library generated with the rat pancreatic tumor cell line BSp73-AS, using the following primers: 5′CTCGGAAGCTTAGCCACCAACCAGCCAAGATGCC3′ and 5′GCCATGAATTCCTAGCCCGCATAGTCAGGAACATCGTATGGGTACATGGCC TCAAACTCGTCGATGCG3′ for full-length ezrin; the 3′ primer for the N-terminal ezrin was 5′GCCATGAATTCCTAGCCCGCATAGTCAGGAATATCGTATGGGTACTGGGCC TTCATCTGCTGCACCTC3′. In addition, the 3′ primer encoded a hemagglutinin marker. The cDNAs were cloned into pcDNA3.1 (Invitrogen, DeShelp). Expression constructs under the control of the CMV promoter which encoded the truncated extracellular CD44 domain were generated as described (Aruffo et al., 1990, Cell 61, 1303-1313). A mutant of the hyaluronate-binding motif was generated as described by Bartolazzi et al. (1994, J. Exp. Med. 180, 53-66). The plasmid encoding the CD44-cytoplasmic moiety was as described by Legg and Isacke (1998, Curr. Biol. 8, 705-708); it was amplified from the bacterial expression clone by means of PCR and inserted into pEBG-3x. In the case of the ezrin-binding-deficient construct mutant, arginine in positions 293 and 294 and lysine in positions 298, 299 and 300 were substituted by alanine (Legg and Isacke, 1998). The expression constructs which encoded the activated oncogenes were as follows: Ras (leu61) (Medema et al., 1991, Mol. Cell. Biol. 11, 5963-5967), Raf BXB (Bruder et al., 1992, Genes Dev. 6, 645-556) obtained from Martin Schwartz, Scripps, MEK-1 DD, subcloned into pcDNA3.1 by Axel Knebel (Mansour et al., 1994, Science 265, 996-997) obtained from Acel Kebel, Dundee). The plasmid Erk-2, which had been equipped with a hemagglutinin marker, was provided by Axel Ullrich (Martinsried).

### 4. Cell Cultures

The schwannoma cell line RT4-D6-P2T and the murine fibroblasts NIH3T3 were purchased from the European Collection of Animal Cell Cultures (Salisbury) and grown in Dulbecco's modified Eagle medium (DMEM; Gibco-BRL, Karlsruhe) supplemented with 10% fetal calf serum (Gibco-BRL). All cells were kept at 37° C. in a humified atmosphere with 5% CO₂.

### 5. Stable and Transient Transfection of the Cells

All transfections were carried out in plates with 6 wells using the liposomal transfection reagent DOTAP (Boehringer Mannheim). The cells were grown under selection with the antibiotic in question in order to obtain stable clones. In order to detect resistance to antibiotics, the following plasmids were cotransfected: pCEP4 (Invitrogen) for hygromycin, pBabe (Invitrogen) for puromycin and pcDNA3.1 (Invitrogen) for neomycin (G418).

### 6. Generation of Doxycyclin-inducible Merlin Cell Lines

The rtTA expression construct pUHD17-1 and the puromycin marker were cotransfected into the rat schwannoma cell line RT4-D6-P2T. Thirty independent puromycin-resistant clones (at 1 μg/ml) were examined for their ability to induce expression of the tet-responsive luciferase reporter construct pUHC13-3 in an assay for transient transfection (Gossen et al., 1995). Three strain clones were obtained in which spontaneous expression was low, but the expression of luciferase was highly inducible upon treatment with doxycyclin. Suitable plasmids encoding merlin or merlin molecule halves were stably transfected, with a neomycin marker, into these strain cell lines. Independent G418 resistant clones (at 500 μg/ml) were selected and the inducibility was verified after addition of doxycyclin. In all in-vitro experiments, the doxycyclin concentration was 1 μg/ml.

### 7. Preparation of Soluble CD44

The expression constructs were transfected COS-1 cells, and soluble protein was purified and quantified as described by Bartolazzi et al., (1994, J. Exp. Med. 180, 53-60).

### 8. In-vivo Tumor Growth

Nude mice were infected subcutaneously into the right flank with 5×10⁵ RT4-D6-P2T schwannoma cells which contained the doxycyclin-inducible merlin construct and had been resuspended in calcium- and magnesium-free PBS. The tumor volumes were determined using calipers. Each data point represents the average tumor volume of four animals +/− standard error. The drinking water for the experimental group in question was treated with doxycyclin (100 μg/ml) one week prior to tumor implantation and then during the entire experiment.

### 9. In-vitro Cell Growth

Soft agar colony test: the cells were treated with trypsin and resuspended in complete medium. 1/10 volume of warmed 3.3% soft agar was added, and each well of a 24-well plate was seeded with 1.25×10³ cells. After the plates had been cooled for two minutes at 40° C., they were incubated, and the colonies were counted after 7 days.

Growth in culture vessels: To determine the growth curves, each well of a 24-well plate was seeded with 5×10³ cells (in FIG. 7: 2.5×10³), and determinations in triplicate were carried out every 24 hours (days). Definition of the growth conditions:

1. Low cell density (=logarithmic or exponential growth) means a cell density of 500 cells per cm² 24 hours after seeding.

2. high cell density (=confluent growth condition) is defined as a cell density of 5×10³ cells per cm² 24 hours after seeding.

### 10. Phosphorylation of Erk

The cells were used to seed 6-well plates at Low density and 24-well plates at high density, with or without 0.8% methylcellulose. The cells remained free from serum for 24 hours and were treated with doxycyclin or left untreated for the last 8 hours. Then, the cells were stimulated for 5 minutes with 5 ng/ml PDGF and subsequently harvested in 2×Laemmli sample buffer, and the cell extracts were separated on a 10% SDS polyacrylamide gel. After the proteins had been transferred to the membrane, immunoblots were prepared with a phospho-Erk-specific antibody, and the bound antibody was detected. As a control for uniform loading of the separating gel with Erk, the membrane was stripped (freed from antibody) and anti-Erk-blotted.

### 11. Immunoprecipitation

For the immunoprecipitation of merlin, the cells were grown to confluence in 10 cm dishes, washed once with ice-cold PBS and then placed on ice and lysed with 1 ml lysis buffer per plate (50 mM Tris pH 7.4, 150 mM NaCl, 3 mM MgCl₂, 0.5% NP-40, 1 mM PMSF, 10 μg/aprotinin ml, 10 μg/ml leupeptin). The DNA was sheared using a gauge 26 needle. The lysate was clarified by centrifugation, and the supernatant was incubated with 5 μg/ml merlin antibody at 4° C. overnight with gentle rotation. The reaction was subsequently treated with 30 μl of protein A agarose (dianova) and left to rotate for a further 3 hours at 4° C. The immune complexes were washed 4 times with cold lysis buffer and separated in 50 μl 2×Laemmli sample buffer. The protein was dissolved in a 10% SDS-PAGE, and immunoblots were generated with a merlin-specific antibody. For the treatment with calf intestine phosphatase (CIP), the lysate was incubated for 1 hour at 30° C. with 1 unit CIP prior to the immunoprecipitation. The coimmunoprecipitation of merlin and CD44 was carried out analogously, except that a different lysis buffer was used (20 mM Tris pH 7.4, 50 mM NaCl, 3 mM MgCl₂, 0.5% NP40). The lysates were preincubated for 2 hours with protein A/G-agarose (Dianova) to remove unspecifically binding components, whereupon 5 μg/ml CD44 antibody 5G8 was added and the reaction was allowed to rotate overnight. The immune complexes were precipitated with protein A/G-agarose. For the coprecipitation with the overexpressed peptides of the cytoplasmid moiety of CD44, the lysates were treated with glutathione agarose (Santa Cruz) or with 5 μg/ml of GST-specific antibody and subsequently with protein A/G-agarose. The immunoprecipitation of HA-labeled Erk was carried out as described above, except that the cells were lysed in RIPA buffer (10 nM Tris pH 7.4, 150 mM NaCl, 1 mM EDTA, 1% Triton X 100, 0.1% SDS, 0.5% deoxycholate, 10 mM NaF, 1 mM vanadate).

### 12. Immunoblotting

Following gel electrophoresis, the proteins were transferred to Immobilon membranes (Millipore, Eschborn). The membranes were incubated for one hour at room temperature in blocking buffer (10% skimmed milk, 0.1% Tween, 10 mM Tris pH 7.6, 100 mM NaCl) and subsequently for another hour or overnight at 4° C. with the primary antibodies in blocking buffer. After three washes, the membranes were incubated for one hour at room temperature with the secondary antibody, developed by enhanced chemiluminescence (Amersham, Brunswick) and visualized by autoradiography.

### DESCRIPTION OF THE FIGURES

### FIG. 1

Tumor suppressor function of merlin (expressed regulaton) in the schwannoma cell line RT4-D6P2T-expressed merlin.

A. Doxycyclin-inducible merlin expression clones. RT4-D6P2T cells were cotransfected stably with a doxycyclin-inducible merlin vector and the r-tet regulator. Three different clones were studied for the expression of merlin. 8 hours after the addition of doxycyclin, the cells were harvested, lysed and subjected to an SDS-PAGE and a Western blotting with antibodies directed against the C terminus (C18) of merlin. The blot which shows endogenous merlin in a vector control clone was exposed approximately 20 times longer.

B. Merlin inhibits tumor growth in vivo. Growth of a subcutaneous tumor following the injection of clone 5₄ cells into nude mice. Where stated, doxycyclin was added to the drinking water. The experiments were also carried out with the other merlin-expressing clones 2₃, 6₃ and 6₇, and similar results were obtained.

C. Merlin reduces the formation of agar colonies. Doxycyclin-inducible clones which expressed either wild-type merlin or merlin moieties, or clones which expressed both the N- and the C-terminal halves of merlin were placed in soft agar. The clones were selected on the basis of their approximately equally pronounced expression. Their expression 8 hours after the addition of doxycyclin was detected by means of Western blotting using antibodies which were directed either against the C terminus (C18; plates 1, 2 and 4) or the N terminus (A19; plates 2 and 4) of merlin. In plate 4, the two lanes on the left represent the Western blot with A19, while the two lanes on the right represent the Western blot of C18. The numbers of colonies per well are plotted. The results were also carried out with clones 2₃, 6₃ and 6₇, and similar results were obtained.

D. N-Terminal ezrin peptide interferes with the function of merlin. Clone 5₄ was stably super-transfected as stated with the merlin and ezrin expression vectors jointly with an expression vector for hygromycin resistance. Hygromycin-resistant clones were placed into soft agar. The expression levels were similar as in C.

E. The agar colony formation assay shows that the clones with mutated merlin lack the tumor suppressor function. The stable transfectants shown (see also Methods) were studied for their ability of forming colonies in soft agar.

FIG. 2

Merlin reduces the cell proliferation in vitro at high cell density. The separately expressed N- and C-terminal peptide halves of merlin reassociate in vivo.

A. Incorporation of ³H-thymidine into schwannoma cells with or without exogenous merlin expression. In each case 3 wells of a 24-well plate were seeded with 2.5×10⁴ schwannoma cells which had previously been transfected with a control vector or a doxycyclin-dependent merlin vector. Where stated, the cells were treated overnight with doxycyclin. The cells were subsequently labeled for 3 hours with 2 μCi/ml ³H-thymidine (Amersham), washed twice with PBS and solubilized in 200 μl of 0.2 M NaOH. The radioactivity incorporated into the cells was determined in a liquid scintillation counter and applied as mean CPM, and the values for the standard error were calculated.

B. The N-terminal halve of merlin is coprecipitated with the C terminus. Cos-1 cells were transfected transiently with the merlin constructs pcDNA3-N-terminal and pcDNA3-C17-terminal. After 36 hours, the cells were lysed with Nonident P-40 buffer (50 mM Tris-HCl ph 7.4, 150 mM NaCl, 0.5% Nonident P-40, 10 μg/ml aprotinin, 1 mM PMSF, 10 μg/ml leupeptin). The proteins were precipitated from the lysates at 4° C. overnight by immunoprecipitation with an antibody with specificity for the merlin C terminus (C-18, Santa Cruz) and subsequently incubated for 3 hours with protein A agarose (Oncogene Science). The immune complexes were obtained by centrifugation and washed 4 times with lysis buffer. The proteins were eluted with 2×Laemmli buffer and studied in a Western blot with an antibody with specificity for the merlin N terminus (A-19, Santa Cruz; 2B, left). To demonstrate the immuno precipitation of the C-terminal merlin fragments, the blot was treated once more, using the antibody C-18, which is specific for the merlin C terminus, as the probe (plate, right).

### FIG. 3

Merlin interferes with signal transduction.

A. PDGF-dependent phosphorylation of Erk. Cells of the merlin-expressing clone 5₄ were placed into culture dishes at high or low density or in methylcellulose (Methocel) and allowed to grow for 24 hours with serum starvation. Where stated, doxycyclin had been present for 8 hours before the treatment with PDGF (5 ng/ml). 5 minutes later, the cells were harvested, and the lysates were subjected to an SDS-PAGE and a Western blotting for phosphorylated Erk and total Erk.

B. Susceptibility of the Ras-, Raf- and MEK-dependent agar colony formation to inhibition by merlin. The inducible clone 5₄ was stably supertransfected with expression constructs which encode constitutively active Ras, Raf or MEK mutants and grown in soft agar.

C. Effects of Ras-, Raf- and MEK-dependent phosphorylation of Erk. The doxycyclin-inducible clone 5₄ was cotransfected in the ratio 5:1 with constructs which encoded the constitutively active Ras, Raf or MEK mutants and with an Erk version which had been provided with a hemagglutinin marker. The cells were plated densely, grown for 24 hours with serum starvation, treated for 8 hours with doxycyclin in the experiments shown and then lysed and subjected to Western blotting as described under A. The experiments A to C were repeated with clones 2₃, 6₃ and 6₇, and similar results were obtained.

### FIG. 4

The inhibition of MEK leads to a higher level of p27^{Kip1} and an increase in hypophosphorylated Rb. Merlin-inducible schwannoma cells were plated onto 6-well plates at a density of 1.5×10⁴ cells per well. 1 μg/ml doxycyclin was added where stated. After 16 hours, an MEK activation inhibitor (UO126; Promega) was added for 2 or 12 hours. 0 means no addition of inhibitor. The cells were harvested and solubilized in 2×Laemmli buffer, and the proteins were separated on a 10% SDS polyacrylamide gel. The proteins were transferred to a membrane, and an immune blot was carried out either with the polyclonal p27^{Kip1} antibody C-19 from rabbit or with the Rb-specific antibody C-15 (Santa Cruz). The actin-specific antibody I-19 was used as loading control.

### FIG. 5

Activation of merlin at high cell density.

A. Growth of the parental RT4-D6P2T cells of clone 5₄ (inducible for merlin expression) and vector control cells in culture dishes (three replications in 24-well dishes). The cells were seeded at low cell density, and the increase in the cell count after 1 to 5 days in the presence or absence of doxycyclin was counted. Plots were established by plotting the cell count versus time. The standard errors are shown. The experiments were also carried out with clones 2₃, 6₃ and 6₇, and similar results were obtained.

B. Immunological detection of the Rb protein of cells of clone 5₄ on the 3rd day of culture in the presence or absence of doxycyclin.

### FIG. 6

High cell density leads to the dephosphorylation of merlin.

A. An equal number of cells which were either in the exponential growth phase or had reached confluence was lysed, and the lysate was subjected to an 8% SDS polyacrylamide gel and to immunoblotting with a merlin-specific antibody (C18). The upper part of FIG. 6A shows clone 5₄ without doxycyclin (to detect endogenous merlin). The lower part shows cells of the same clone 8 hours after addition of doxycyclin (exposure time of the film approximately 30 times less).

B. Immune blot of immunoprecipitates. The band which migrates more slowly disappears upon digestion with calf intestinal phosphatase (CIP) prior to application to the gel.

### FIG. 7

The function of merlin depends on interaction with the transmembrane protein CD44.

A. Doxycyclin-induced merlin is coprecipitated with the CD44-specific antibody (5G8). Confluent cells of clone 5₄ were treated for 8 hours with doxycyclin and then lysed. The lysates were subjected to an SDS-PAGE, either directly or after treatment and isolation of the proteins with protein A/G Sepharose beads with or without previous addition of an antibody (CD44, 5G8). Immune blots were prepared sequentially with merlin- and ezrin-specific antibodies. The anti-ezrin-antibodies crossreact with moesin.

B. The sequestration of merlin by binding to overexpressed soluble CD44-cytoplasmic-moiety-homologous peptides nullifies the function of merlin. The 5₄ clone which expresses doxycyclin-inducible merlin was transfected stably with expression constructs which encode either the CD44 cytoplasmic moiety in its wild type form or the form of a mutant, both fused to GST. The mutated form was chosen owing to its inability to bind ezrin (see text and methods). Subclones with high expression levels (see Western blot with CD44-cytoplasmic-moiety-specific antibodies) were selected. Their colony forming ability in soft agar in the presence or absence of doxycyclin was determined.

C. and D. Coprecipitation of merlin and ezrin with the overexpressed cytoplasmic moiety of CD44. C. The cells of B were plated at high density. 8 hours after the addition of doxycyclin, the cells were lysed, and the GST fusion peptides were concentrated by means of GSH agarose. D. Doxycyclin-treated cultures at low cell density were lysed, and the GST fusion peptides were precipitated with GST-specific antibodies. The immune blots with antibodies with specificity for merlin, ezrin and CD44 cyctoplasmic moiety are shown. It must be noted that less protein was harvested from cells in the log phase, for technical reasons.

### FIG. 8

CD44 is both a tumor suppressor and an oncogene. The function of CD44 depends on ligands and probably on the tumor-specific regulation and tumor-specific mutations. In its suppressor function, CD44 activates merlin. The scheme shows the smallest isoform, which is CD44s. Larger splice variants are probably also capable of activating merlin. Interference with the signal transduction by merlin blocks the activation, by growth factors and by the Ras-Raf-pathway, of promoters such as, inter alia, the CD44 promoter. Moreover, the Ras-dependent incorporation of exons is interfered with, thus reducing the production of larger CD44 variants. In growth mode, both CD44s and the CD44 splice variants act as a platform for the presentation of growth factors and other molecules which are connected to growth. The scheme only shows a large CD44 variant (the field divided into rectangles represents variable exon sequences). The oligomerization of the variant splice form is also shown. All CD44 variants which bear sequences encoding at exons v6 and v7 tend to clustering (symbolized by arrows) probably thereby enhancing the platform function.

### FIG. 9

Model of the action of CD44 under conditions of exponential and confluent growth. Specific ligands determine two functional states of CD44, which affect the cytoplasm complexes. GF=growth factor, MMP=metalloprotease, PP=protein phos-phatase. The gray field means that additional components are probably also associated with the CD44-bound complexes.

### FIG. 10

Ligand-dependent activity of merlin, and blocking of the activity.

A. The soluble extracellular domain of CD44 sequestrates different ligands of exponentially growing and confluent cells. In each case 3 wells of a 24-well plate were seeded in triplicate with cells of clone 5₄, and the cells were treated with doxycyclin in order to induce the expression of merlin. On day 1 (logarithmically growing cells) and on day 3 (confluent cells), 10 ng/ml of soluble extracellular CD44 domain, either of the wild type (solCD44 or solCD44wt) or of the glycosaminoglycan-binding mutant (solCD44mut), were added. As the control, cells were treated in the same manner without doxycylin (since the addition of solCD44 was ineffective, only one group of data is shown).

B. Induced dephosphorylation of merlin in exponentially growing cells as response to solCD44wt. Detection of merlin in lysates of exponentially growing cells of clone 5₄ which had been treated for 8 hours with doxycyclin, by means of immune blot. Comparison of the cells in the absence of solCD44wt or 5 minutes after addition of 10 ng/ml solCD44wt. Gel resolution as in FIG. 4.

C. Hyaluronidase destroys the ligand responsible for the activation of merlin in confluent cells, while the sequestration of a ligand of exponentially growing cells nullifies blocking of the activation of merlin. Cells of clone 5₄ were plated at high or low cell density, left to grow for 24 hours with serum starvation and treated during the last 8 hours with doxycyclin (+) or left untreated (−). Where stated, the cells were treated for 2 hours with hyaluronidase (HAase, 5 U/μl), for 10 minutes with solCD44wt and/or for 5 minutes with PDGF (5ng/ml). The proteins of the lysates were separated by means of SDS-PAGE, and immune blots were carried out with phospho-Erk and Erk-specific antibodies. Clone 6₃ gave the same results as all the experiments in FIG. 6.

### FIG. 11

The CD44 ligand hyaluronate imitates high cell density and leads to the activation of merlin.

A. Induced dephosphorylation of merlin in exponentially growing cells as a response to the addition of hyaluronate. The experiment was carried out as described for FIG. 6A, except that hyaluronate (HA) was added in place of solCD44wt.

B. Hyaluronate delays growth. In each case 3 wells of a 24-well plate were seeded in triplicate with cells of clone 5₄. Doxycyclin and hyaluronate (100 μg/ml) were added as stated.

C. Hyaluronate-activated merlin blocks the PDGF-dependent phosphorylation of Erk. Cells were grown at low cell density and subsequently treated with doxycyclin with serum starvation cells as shown in FIG. 6C and assayed for the PDGF-dependent phosphorylation of Erk. Where stated, HA (100 μg/ml) was present for 10 minutes and PDGF (5 ng/ml) was present for 5 minutes. Experiments A to C were also carried out with clone 6₃, and similar results were obtained.

### FIG. 12

The hyaluronan-dependent activation of merlin in NIH3T3 cells reduced their growth rate. The cell division cycle inhibitor p27^{Kip1} increased both by addition of hyaluronate and by addition of solCD44s.

A. Growth rate of NIH3T3 cells after addition of hyaluronan. 24-well plates were seeded in triplicate with NIH3T3 cells at a density of 0.5×10⁴ cells. Where stated, the culture medium was treated the next day with 100 μg/ml hyaluranon (Healon). The cell counts were determined on each of the following 3 days.

B. Induction of p27^{Kip1} by treatment with hyaluronan. NIH3T3 cells were plated in 6-well plates at a density of 1.5×10⁴ cells. The next day, they were treated either with 100 μg/ml of hyaluronan or with 10 ng/ml solCD44s, and the plates were incubated for 30 minutes. The cells were harvested in 2×Laemmli sample buffer, and an immune blot with a polyclonal, p27^{Kip1}-specific antibody from rabbit was carried out (C-19, Santa Cruz). A polyclonal actin-antibody from goat (I-19, Santa Cruz) was used as loading control.

We claim:

1. A method for identifying compounds which modify the activity of the intracellular tumor suppressor gene nf2, where the activity of the protein NF2, which is encoded by the gene nf2, is modified by an extracellular interaction of the compounds with the cell surface protein CD44.

2. A method as claimed in claim 1, characterized in that the expression of the gene nf2 or the activity of the protein NF2 is increased.

3. A method as claimed in claim 1 or 2, characterized in that NF2 is activated by dephosphorylation of the protein.

4. A method as claimed in any of claims 1 to 3, characterized in that
a) a cell culture is equipped with a dominant oncogene,
b) this “oncogenic” cell culture is additionally equipped with a gene construct which contains a promoterless reporter gene under the control of an Ras-dependent promoter,
c) the cell culture thus obtained is provided with a compound which, owing to interaction with a cell surface protein, is potentially capable of increasing the intracellular activity of NF2,
d) a substance which can be converted by the expression product of the reporter gene is added to the cell culture thus treated,
e) the substance added in d) is, if appropriate, removed,
f) the cell culture thus treated is equipped with a suitable culture medium in order to multiply the cells, and
g) a compound of c) is indeed identified as one which increases the intracellular activity of NF2 by the fact that the cells multiply and/or the expression product of the reporter gene is detected specifically.

5. A method as claimed in any of claims 1 to 4, characterized in that the gene RasV12 is employed as the dominant oncogene in step a).

6. A method as claimed in any of claims 1 to 5, characterized in that the Ras-dependent promoter employed is the promoter of the c-fos gene or of a collagenase gene.

7. A method as claimed in any of claims 1 to 6, characterized in that a gene construct comprising a promoterless thymine kinase gene under the control of an Ras-dependent promoter is employed in step b).

8. A method as claimed in any of claims 1 to 7, characterized in that gancyclovir is employed in step d).

9. A method as claimed in any of claims 1 to 8, characterized in that a gene construct comprising a promoterless reporter gene is employed in step b), a CD44 exon, preferably the CD44 exon v5, being integrated into the coding region of the reporter gene.

10. A method as claimed in claim 9, characterized in that a promoterless luciferase gene or a promotorless green fluorescent protein is employed as reporter gene.

11. A method as claimed in any of claims 1 to 10, characterized in that, owing to an increased NF2 activity caused by the compound added in step c), the CD44 exon is excised specifically from the mature mRNA of the reporter gene and the expression product of the reporter gene is detected specifically.

12. A method as claimed in any of claims 1 to 11, characterized in that a rat schwannoma system comprising clones of RT4-D6P2T cells is preferably used, these clones comprising an expression vector encoding a reverse-tet repressor and also a merlin cDNA plasmid under the control of a tet repressor recognition sequence.

13. A vector for use in a method as claimed in any of claims 1 to 12, characterized in that it comprises a promoterless reporter gene under the control of an Ras-dependent promoter and additional structures responsible for NF2-dependent, targeted splicing of exons from the derived mature mRNA of the reporter gene.

14. A compound identified by a method as claimed in any of claims 1 to 12.

15. A compound as claimed in claim 14, characterized in that it is the CD44-specific antibody IM7 or KM81.

16. A compound as claimed in claim 14, characterized in that it is a low-molecular-weight chemical compound, preferably other than hyaluronic acid.

17. A compound as claimed in any of claims 14 to 16, characterized in that it binds specifically to a sequence of a cell surface protein.

18. The use of a compound as claimed in any of claims 14 to 17 for the preparation of compositions for treating carcinomas.
